(19) United States
(12) Patent Application Publication
(10) Pub. No.: US 2006/0222689 A1
(43) Pub. Date: Oct. 5, 2006

(54) Skin care compositions and methods

(75) Inventor: TongHo Lin, Taipei City (TW)

Correspondence Address: OSHA LIANG L.L.P., 1221 MCKINNEY STREET, SUITE 2800, HOUSTON, TX, 77010, US

(73) Assignee: Lotus Pharmaceutical Co., Ltd., Taipei (TW)

(21) Appl. No.: 11/446,051

(22) Filed: Jun. 2, 2006

Related U.S. Application Data
(63) Continuation-in-part of application No. 10/811,420, filed on Mar. 26, 2004.

(30) Foreign Application Priority Data
May 16, 2003 (CN) 03136028.9
May 16, 2003 (WO) PCT/CN03/00358

(51) Int. Cl. A61K 31/714; A61K 31/525; A61K 31/51; A61K 31/4415; A61K 31/455; A61K 31/375; A61K 31/355; A61K 9/70; A01N 31/14

(52) U.S. Cl. 424/443; 424/448; 514/52; 514/251; 514/276; 514/350; 514/355; 514/474; 514/723; 514/458

(57) ABSTRACT

A composition for local application includes: vitamin C in an amount from about 1% to 45% w/w; vitamin B complex in an amount from about 1% to 5% w/w; carotene in an amount from about 0.1% to 3% w/w; and vitamin E in an amount from about 2% to 90% w/w. The composition may further includes 0.1-2% fragrance, 1-5% w/w thickener, 1-8% w/w surfactant.

### CROSS-REFERENCE TO RELATED APPLICATIONS

This is a Continuation-In-Part of U.S. patent application Ser. No. 10/811,420, filed on Mar. 26, 2004, which claims priority of Chinese Patent application Serial No. 03136028.9 filed May 16, 2003 and PCT Application Serial No. PCT/CN03/00358 filed May 16, 2003. These applications are incorporated by reference in their entireties.

### STATEMENT REGARDING FEDERALLY SPONSORED RESEARCH OR DEVELOPMENT

Not applicable.

### BACKGROUND OF INVENTION

1. Field of the Invention

The invention relates generally to compositions for topical applications. Particularly, the invention relates to compositions for skin care.

2. Background Art

Human skin structure consists of epidermis, dermis, subcutaneous fatty tissue, sebaceous gland, sweat glands, and hairs. The thick layer under the epidermis is the dermis. The hairs are surrounded by hair follicles, which have associated sebaceous glands. The sebaceous glands secrete sebum, which will be secreted to skin surface via the hairs and hair follicles. The secreted sebum becomes fatty film on the skin. The fatty film attaches to the surface of the skin and protects the skin. Skin types may be classified according to the quantity of sebaceous glands as oily skin, dry skin, and mixed-type skin.

Acne, also commonly known as comedo or zit, is a chronic inflammation in the hair follicle and the sebaceous gland. It is usually found on the forehead, around the nose, and on the cheeks. It can also be found on any part of the body with hair follicles, such as the back, chest, and thigh. Depending on the symptoms, acne can be classified as acne-type, inflammatory and red swelling type, and cyst-type. When gonads of adolescents mature, androgenic hormone in the testes and ovaries increases Androgenic hormone stimulates the sebaceous glands. As a result, the sebaceous glands become hypertrophic and secrete abundant sebum, which accumulates around the sebaceous glands and follicles. The accumulated sebum may become infected by bacteria, leading to inflammation of the sebaceous glands and follicles.

Typically, acne with a white or black head forms first; it subsequently becomes papule, pustule, node and cyst after bacterial infection. Finally, a zit is formed. When too much androgenic hormone is secreted, redness, tickle, heat, burning sensation, and skin peeling may occur around the T area of the face or the skin under the eyes. This sometimes leads to seborrheic dermatitis, which is characterized by the appearance of visible microvessels.

To prevent these problems, topical preparations may be needed to clean the skin, kill the bacteria, prevent water evaporation, and improve skin moisture retention. Examples of topical preparations for these purposes include a cream containing sodium chloride for mollifying and soothing the skin disclosed in U.S. Pat. No. 3,574,854, and a skin-care composition containing mineral salts disclosed in German patent publication No. DE 3,327,840. Furthermore, U.S. Pat. No. 3,859,436 disclosed a glucose mixture for soothing skin, and U.S. Pat. No. 3,777,597 disclosed a shaving aqueous solution comprising glucose.

Sodium Chloride, especially 0.9% sodium chloride solution, i.e., saline, is the main composition in maintaining the osmotic pressure of body fluids. A solution containing a higher concentration of sodium chloride cannot be absorbed by the skin. Instead, it will irritate the skin mucosa and results in dehydration. Thus, topical preparations containing sodium chloride may irritate skin or kill bacteria. However, sodium chloride can hardly prevent water evaporation from skin or improve skin moisture retention.

Glucose may increase glycogen, ensure cellular functions, improve metabolism by functioning as a nutritional agent. In addition, it may function as a detoxification agent. However, these effects cannot be achieved by applying the topical preparations containing glucose to the skin. Instead, glucose must be administered orally, intravenously and intramuscularly to have these effects.

Various pharmaceutically active topical preparations include skin absorption enhancing agents, such as di-butyl dihaxanoate (DBA) or its mixture with isopropyl myristate (IPM). IPM is a known skin permeation enhancer for use with topical preparations. However, the efficacy of the mixture of DBA and IPM as skin absorption enhancer is not known.

There have been many therapeutic agents and cosmetics for treating various skin conditions, such as hydrocortisone for treating atopic dermatitis with titillate and erythema, sulconazole nitrate for treating mycotic infection in the skin, tretinoin for treating light-induced skin aging, and 5-fluorouracil for treating psoriasis and skin cancer. Therapeutic agents for use in treating dermatologic diseases usually include permeation enhancers, such as dimethyl sulfoxide (DMSO), dimethyl formamide, methyldecyl sulfoxide (U.S. Pat. No. 3,527,864), dimethyl acetamide (U.S. Pat. No. 3,472,931) and N-alkyl-2-pyrrolidone (U.S. Pat. No. 3,696,516). However, the above-described permeation enhancers have certain drawbacks. For example, dimethyl sulfoxide has an unpleasant odor and body odor, can burn the skin and induce erythema on skin, can reduce the transparency of crystalline-skin, and can even cause tissue necrosis in animals. (Martindale, The Extra Pharmacopoeia, pages 1461-1463, 27^{th} Ed., 1977). Dimethyl formamide and dimethyl acetamide can also burn the skin and induce erythema on skin.

Moreover, Trebosc et al. (U.S. Pat. No. 5,030,451) disclosed a cosmetics composition containing improved derivatives of caffeine as active ingredients. The formulation has excellent long-acting “lipolytic” properties and has proven very effective in weight loss programs and in the treatment of cellulite. An anti-cellulite composition containing methylsilanol theophyllin acetate alginate and methylsilanol mannuronate is disclosed by Mausner in U.S. Pat. No. 5,215,759. In U.S. Pat. No. 5,051,449, Kligman disclosed a method of reducing cellulite by locally applying retinoid to skin. Kligman reported that the treated subjects have a thickened dermis and an increased number of new blood vessels, with a moderate to noticeable degree of improvement in the pinching test.

Topical aminolevulinic acid-photodynamic therapy for the treatment of acne vulgaris is disclosed in U.S. patent application No. 20020099094 A1. U.S. patent application No. 20020061855 A1 disclosed a composition for treating acne comprising water and glycol, and U.S. patent application No. 20010056071 A1 disclosed a composition for treating acne comprising resveratrol (3,4′,5-trihydroxy-trans-stilbene), melatonin, Vitamins D, E, and A. U.S. Pat. No. 5,710,177 discloses the use of biotin, biotin esters, hydroxycarboxylic acids, and antioxidants. U.S. Pat. No. 4,938,960 discloses the use of vitamin B complexes, vitamin E, vitamin C, and phospholipids for skin treatments.

The above described skin treatment preparations are claimed to be effective in treating acnes, lipolytic and various skin treatments. These preparations all contain vitamin A acid.

In addition, Shapiro, S. S. and Saliou, C. described that vitamin A, vitamin D and their derivatives in combination with vitamin C, vitamin E, and coenzyme Q could improve skin conditions and cure acne (Nutrition 2001, Vol. 17(10), pages 839-844). Vitamin A acid (i.e., Tretinoin or Retinoids) is a derivative of Vitamin A. Some of its major pharmaceutical effects include removing cutin on the epiderm, alleviating occlusion of the pores, reducing wrinkles and improving blood circulation around the face, decreasing scar of pigment, and preventing skin keratinization, promoting refreshing and sloughing off of epithelium cells, preventing synthesis of keratin, as well as preventing the formation of blister on the face. However, most Vitamin A acid-containing products may sensitize skin to the light, and overuse of these products may cause side effects, such as dry, red swelling, itching dermatitis, and etc.

In addition to acnes, human skins also suffer from aging and wrinkles. Dryness of skin accelerates the aging of skin and generates wrinkles. Therefore, it is desirable to keep skin properly hydrated. Two mechanisms contribute to skin hydration: natural moisturizing factor (NMF) inside the cuticles helps to maintain the skin hydration; and sebum secreted by the sebaceous glands helps to prevent loss of moisture on the external layer of skin. These protective mechanisms would gradually lose their effects due to various external factors. Once these protective mechanisms lose their effects, regular skin may become dry skin. Extra dry skin may result in skin peeling, lack of softness, infection, and chap. Therefore, skin moisturizing products are often used to help maintain and improve skin hydration.

Various natural and synthetic skin moisturizing agents are known in the art. For example, U.S. Pat. No. 4,837,019 issued to Georgalas et al. discloses compositions for counteracting moisture loss and promoting the healing of burned skin. The moisturizing components in these compositions include polyglyceryl methacrylate, glycerine, allantoin, panthenol, amino acid complex, and fibronectin. These components are present in an amount of 2-30% of the total composition.

U.S. Pat. No. 4,863,725 issued to Deckner et al. discloses moisturizing compositions, which are clear, oil-free and, therefore, non-greasy to the skin and touch. The major moisturizing components of these compositions comprise glycerol and acrylic or methacrylic polymers (polyglyceryl methacrylate), one or more skin-feel enhancers, which is preferably a polyol, one or more preservatives, and water as a carrier. These moisturizer compositions may also include one or more thickeners, one or more skin conditioning agents, one or more astringents and/or colorants and/or fragrances.

U.S. Pat. No. 5,302,377 issued to Pereira et al. discloses lotions containing a fatty alkoxylate ester, which is a diester or triester of an aliphatic or aromatic dicarboxylic or tricarboxylic acid. These lotions can be used as an emollient to smooth and soften skin. U.S. Pat. No. 6,770,286 B1 issued to Berry discloses a patch for treating dry skin. The moisturizing components of the patch is based on polyvinyl alcohols.

U.S. Pat. No. 5,834,445 issued to Sikorski et al. discloses a topical composition containing carotene and vitamin E capable of reaching langerhans cells to improve local immunity. However, due to the heavy color of carotene and its high lipid-solubility, there are significant technical difficulties in its applications in some cosmetics and lotions.

While the prior art skin care compositions are effective in treating acnes or improving skin hydration, there is still a need for better skin care products that can treat acnes and/or improving skin hydrations.

### SUMMARY OF INVENTION

One aspect of the invention relates to compositions for skin care. A composition in accordance with one embodiment of the invention includes: vitamin C in an amount from about 1% to 45% w/w; vitamin B complex in an amount from about 1% to 5% w/w; carotene in an amount from about 0.1% to 3% w/w; and vitamin E in an amount from about 2% to 90% w/w. The composition may further include 0.1-2% w/w fragrance, 1-5% w/w thickener, 1-8% w/w surfactant.

Other aspects of the invention relate to methods of treating a skin condition. The skin condition may be one or more selected from acnes, zits, comedo, and skin dryness.

Other aspects and advantages of the invention will be apparent from the following description and the appended claims.

### BRIEF DESCRIPTION OF DRAWINGS

FIG. 1 shows a protocol used to test the efficacy of a composition in accordance with one embodiment of the invention.

FIG. 2 shows results of skin hydration improvement on foreheads by using a composition (Lo-108) in accordance with one embodiment of the invention.

FIG. 3 shows results of skin hydration improvement on cheeks by using a composition (Lo-108) in accordance with one embodiment of the invention.

FIG. 4 shows results of skin hydration improvement on foreheads by using a composition (Lo-107) in accordance with one embodiment of the invention.

FIG. 5 shows results of skin hydration improvement on cheeks by using a composition (Lo-107) in accordance with one embodiment of the invention.

### DETAILED DESCRIPTION

This invention relates to compositions for skin-care, treatment, and moisturization. A composition in accordance with embodiments of the invention can be applied onto local areas of skin, such as arms and face, to moisturize skin and to treat skin dryness, as well as common skin conditions such as acne, zit, comedo, or dryness.

Compositions according to embodiments of the present invention mainly comprise vitamins, such as vitamin C, vitamin B complex, carotene, and vitamin E. In addition, compositions of the invention may also include a fragrance, a thickening agent, surfactants, and other pharmaceutically acceptable excipients. Pharmaceutically acceptable excipients are additives that are compatible with the pharmaceutically active ingredients. Because main components of these compositions are vitamins, there is less possibility of toxicity even after long-term use.

The amounts of vitamins needed by human body are minimal, but the functions of vitamins are essential. Vitamins cannot be synthesized by human body and, therefore, need to be taken in from outside sources. The major functions of vitamin C in the body include: anti-oxidation (e.g., preventing the formation of peroxylipids), facilitating the formation of collagen, assisting various enzymes, retarding aging of cells, improving blood circulation, and reducing melanin. Therefore, vitamin C is commonly believed to contribute to skin's regeneration, to prevent the generation of melanin, and to enhance the immunity.

Vitamin B complex includes a number of related vitamins, such as vitamin B₁ (thiamin), vitamin B₂ (riboflavin), Niacin (sometimes referred to as vitamin B₃), vitamin B₅ (pantothenic acid), vitamin B₆ (pyridoxal), folic acid, vitamin B₁₂ (cobalamin), biotin, and etc. Vitamin Bs mainly function as coenzymes. Their functions include facilitating the oxidation of glucose and energy release from fats and proteins to maintain the normal functions of the nervous system. Vitamin Bs are essential for the growth and regeneration of cells, the generation of erythrocyte, and the synthesis of nucleoprotein and myelin. They can also activate the folic acid coenzyme to facilitate the generation of erythrocytes. As used herein, “vitamin B complex” refers to a mixture that includes at least two different kinds of vitamin Bs. That is, vitamin B complex does not have to include all vitamin Bs.

Vitamin E is an oxidation inhibitor, and it can also inhibit platelet aggregation. As an anti-oxidant, vitamin E can prevent the oxidation of cell membranes. For example, vitamin E can prevent oxidation of erythrocyte membranes, and, therefore, protect the erythrocytes and prevent anemia. In addition to maintaining the integrity of cell membranes, vitamin E can also enhance the function of linolenic acid, protect the structure and function of muscles and nerve tissues, and increase capillary blood flow to improve blood circulation. Although Vitamin E has the above-described activities, most commercially available vitamin E-containing skin care products, such as Walgreen, L'oreal Future-E®, or Jasom Natural Cosmetics, have not been shown to have anti-acne effects.

Skin care compositions according to embodiments of the present invention comprise well-known ingredients, such as vitamin C, vitamin B complex, carotene, and vitamin E. In addition, these compositions may also contain fragrance, thickening agents, and surfactants. These compositions are found to be efficient skin moisturizers and can prevent and treat acnes. These effects are due to proper combinations of these ingredients because the individual components are not known to have such effects. Compositions of the invention include mainly common vitamins and are free of vitamin A acid. Therefore, these compositions do not have risks of osteoporosis even after long-term use.

Although U.S. Pat. No. 5,834,445 discloses that carotene and vitamin E can act on Langerhans cells to enhance local immune responses. However, carotene has an intense color and is hydrophobic. It is technically difficult to mix carotene into creams, lotions or cosmetics preparations.

Main components of a composition in accordance with embodiments of the invention, for example, may comprise about 1-45% W/W vitamin C, about 1-5% W/W vitamin B complex, about 0.1-5% W/W carotene, and about 2-90% W/W vitamin E. In addition, these compositions may include 0.1-2% fragrance, 1-5% thickening agents, 1-8% surfactants, and an appropriate amount of water. The main components of preferred compositions may comprise about 4-15% W/W vitamin C, about 1-3% W/W vitamin B complex, about 1-3% W/W carotene, and about 20-65% W/W vitamin E. The percentages as used herein are by weight, relative to the total weight of the composition, i.e., weight of a component/weight of the composition (W/W).

Compositions of the invention comprise the above mentioned ingredients and do not contain vitamin A acid. These compositions are skin care compositions for maintaining skin moistures or treating acnes and zits. These compositions, which comprise vitamin C, vitamin complex B, carotene, vitamin E, and optionally fragrance, thickening agents, or surfactants, have effects that are different from the known effects of the individual vitamin components. These compositions are for topical application as antioxidant and for treating acnes, zits, comedo. These compositions can also maintain skin moistures.

When needed, compositions of the invention may include other pharmaceutically acceptable excipients, carriers, or diluents. These compositions may be in a form of ointments, creams, cleansing agents, or patches for local application. Pharmaceutically acceptable excipients, for example, may include gum Arabic or starch, and carriers may include starch or talc. Such compositions may include surfactants selected from, for example, sodium lauryl sulfate, docusate sodium, Tween® 80, Tween® 60, Tween® 20. Binders, such as starch, and thickening agents such as sodium carboxymethylcellulose (CMC), hydroxypropyl cellulose, mydroxypropyl methyl cellulose, acacia, and povidone, may be added to the compositions to facilitate their preparation as desired. Compositions or skin care products of the invention may be prepared in any form using conventional techniques known in the art. Buffer salt such as phosphate may also be added to adjust the pH value to a proper range, typically around pH 7.4. Permeation enhancers or extracts from natural plants such as licorice root may also be added according to methods known in the art. In addition, fragrances and/or coloring agents may be added to render the compositions or skin care products more pleasing to the users.

The following examples are provided to further illustrate the present invention.

### EXAMPLES

### Example 1

### Lo-107

| Formulation | Lo-107 |
|---|---|
| Vitamin E | 25% |
| Vitamin C | 20% |
| Vitamin B-complex | 2% |
| Carotene | 0.1% |
| Fragrance | 2% |
| Surfactant (Tween ® 60) | 8% |
| Thickener (Na CMC) | 1% |
| Distilled water | 41.9% |

In this particular example, 20% w/w vitamin C and 2% w/w B-complex are first dissolved in a small amount of distilled water. A separate solution, comprising 0.1% w/w carotene, 25% w/w vitamin E, 2% w/w flagrance, 8% w/w surfactant (Tween® 60), and 1% w/w thickener (Na CMC), is also prepared. The two solutions are then combined and add a sufficient amount of distilled water (to reach 41.9%).

### Example 2

### Lo-108

| Formulation | Lo-108 |
|---|---|
| Vitamin E | 65% |
| Vitamin C | 4% |
| Vitamin B-complex | 1% |
| Carotene | 1% |
| Fragrance | 1% |
| Surfactant (Tween ® 60) | 6.5% |
| Thickener (Na CMC) | 4% |
| Distilled water | 17.5% |

In this particular example, 4% w/w vitamin C and 1% w/w B-complex are first dissolved in a small amount of distilled water. A separate solution, comprising 1% w/w carotene, 65% w/w vitamin E, 1% w/w flagrance, 6.5% w/w surfactant (Twee® 60), and 4% w/w thickener (Na CMC), is also prepared. The two solutions are then combined and brought up to 100% final weight by adding sufficient amount of distilled water.

### Example 3

### Lo-109

| Formulation | Lo-109 |
|---|---|
| Vitamin E | 40% |
| Vitamin C | 10% |
| Vitamin B-complex | 3% |
| Carotene | 0.5% |
| Fragrance | 1% |
| Surfactant (Sodium Lauryl Sulfate) | 8% |
| Thickener (gum Arabic) | 5% |
| Distilled water | 32.5% |

In this particular example, 10% w/w vitamin C and 3% w/w B-complex are first dissolved in a small amount of distilled water. A separate solution, comprising 0.5% w/w carotene, 40% w/w vitamin E, 1% w/w flagrance, 8% w/w surfactant (sodium lauryl sulfate), and 5% w/w thickener (gum Arabic), is also prepared. The two solutions are then combined and brought up to 100% final weight by adding sufficient amount of distilled water.

### Example 4

### Lo-110

| Formulation | Lo-110 |
|---|---|
| Vitamin E | 20% |
| Vitamin C | 30% |
| Vitamin B-complex | 4% |
| Carotene | 4% |
| Fragrance | 2% |
| Surfactant (Sodium Lauryl Sulfate) | 8% |
| Thickener (gum Arabic) | 5% |
| Distilled water | 45% |

In this particular example, 30% w/w vitamin C and 4% w/w B-complex are first dissolved in a small amount of distilled water. A separate solution, comprising 4% w/w carotene, 20% w/w vitamin E, 2% w/w flagrance, 8% w/w surfactant, and 5% w/w thickener, is also prepared. The two solutions are then combined and brought up to 100% final weight by adding sufficient amount of distilled water.

### Example 5

### Lo-122

| Formulation | Lo-122 |
|---|---|
| Vitamin E | 85% |
| Vitamin C | 2% |
| Vitamin B-complex | 1% |
| Carotene | 1% |
| Fragrance | 0.1% |
| Surfactant (Sodium Lauryl Sulfate) | 3% |
| Thickener (gum Arabic) | 2% |
| Permeation enhancer | 0.5% |
| Distilled water | 5.4% |

In this particular example, 2% w/w vitamin C and 1% w/w B-complex are first dissolved in a small amount of distilled water. A separate solution, comprising 1% w/w carotene, 85% w/w vitamin E, 0.1% w/w flagrance, 3% w/w surfactant, 0.5% w/w permeation enhancer, and 2% w/w thickener, is also prepared. The two solutions are then combined and brought up to 100% final weight by adding sufficient amount of distilled water.

### Example 6

### Lo-130

| Formulation | Lo-130 |
|---|---|
| Vitamin E | 40% |
| Vitamin C | 10% |
| Vitamin B-complex | 3% |
| Carotene | 2% |
| Fragrance | 2% |
| Surfactant (Sodium Lauryl Sulfate) | 10% |
| Thickener (gum Arabic) | 5% |
| Distilled water | 28% |

In this particular example, 10% w/w vitamin C and 3% w/w B-complex are first dissolved in a small amount of distilled water. A separate solution, comprising 2% w/w carotene, 40% w/w vitamin E, 2% w/w flagrance, 10% w/w surfactant, and 5% w/w thickener, is also prepared. The two solutions are then combined and brought up to 100% final weight by adding sufficient amount of distilled water.

### Example 7

### Lo-18

| Formulation | Lo-18 |
|---|---|
| Vitamin E | 90% |
| Vitamin C | 1% |
| Vitamin B-complex | 1% |
| Carotene | 1% |
| Fragrance | 0.5% |
| Surfactant (Tween ® 60) | 3% |
| Thickener (Na CMC) | 3% |
| Distilled water | 0.5% |

In this particular example, 1% w/w vitamin C and 1% w/w B-complex are first dissolved in a small amount of distilled water. A separate solution, comprising 1% w/w carotene, 90% w/w vitamin E, 0.5% w/w flagrance, 3% w/w surfactant, and 3% w/w thickener, is also prepared. The two solutions are then combined and brought up to 100% final weight by adding sufficient amount of distilled water.

### Example 8

### Lo-27

| Formulation | Lo-27 |
|---|---|
| Vitamin E | 30% |
| Vitamin C | 45% |
| Vitamin B-complex | 1% |
| Carotene | 2% |
| Fragrance | 2% |
| Surfactant (Tween ® 60) | 8% |
| Thickener (Na CMC) | 1% |
| Permeation enhancer | 0.1% |
| Distilled water | 10.9% |

In this particular example, 45% w/w vitamin C and 1% w/w B-complex are first dissolved in a small amount of distilled water. A separate solution, comprising 2% w/w carotene, 30% w/w vitamin E, 2% w/w flagrance, 8% w/w surfactant, 0.1% w/w permeation enhancer, and 1% w/w thickener, is also prepared. The two solutions are then combined and brought up to 100% final weight by adding sufficient amount of distilled water.

### Example 9

### Lo-22

| Formulation | Lo-22 |
|---|---|
| Vitamin E | 80% |
| Vitamin C | 4% |
| Vitamin B-complex | 2% |
| Carotene | 3% |
| Fragrance | 0.1% |
| Surfactant (Tween ® 60) | 3% |
| Thickener (gum Arabic) | 2% |
| Distilled water | 5.9% |

In this particular example, 10% w/w vitamin C and 3% w/w B-complex are first dissolved in a small amount of distilled water. A separate solution, 2% w/w carotene, 40% w/w vitamin E, 2% w/w flagrance, 10% w/w surfactant, and 5% w/w thickener, is also prepared. The two solutions are then combined and brought up to 100% final weight by adding sufficient amount of distilled water.

### Example 10

### Lo-39

| Formulation | Lo-39 |
|---|---|
| Vitamin E | 30% |
| Vitamin C | 10% |
| Vitamin B-complex | 5% |
| Carotene | 2% |
| Fragrance | 2% |
| Surfactant (Sodium Lauryl Sulfate) | 10% |
| Thickener (Na CMC) | 5% |
| Permeation enhancer | 1.5% |
| Distilled water | 34.5% |

In this particular example, 10% w/w vitamin C and 5% w/w B-complex are first dissolved in a small amount of distilled water. A separate solution, 2% w/w carotene, 30% w/w vitamin E, 2% w/w flagrance, 10% w/w surfactant, and 5% w/w thickener, is also prepared. The two solutions are then combined and brought up to 100% final weight by adding sufficient amount of distilled water.

Various formulation methods known in the art may be used to prepare compositions of the invention. For example, water-soluble components may be dissolved in a small amount of water, and water-insoluble components may be dissolved in a suitable solvent. Then, the two solutions are mixed, and the mixture is adjusted to the proper volume or weight. For example, a formulation similar to Lo-108 shown in Table 1 may be prepared as follows. Vitamin C and vitamin B complex were dissolved in a small amount of distilled water, while carotene, vitamin E, a fragrance, a surfactant, and a thickening agent were mixed in a separate container, with the aid of a solvent (such as alcohol, glycerol, etc.), if necessary. Then, these two are mixed, and a sufficient amount of distilled water is added therein to the required amount. If other excipients, carriers, or additives are desired, they may be added to either solution before mixing or added after the two solutions are mixed.

Compositions of the invention may be prepared in any form commonly used for skin care products, including creams, ointments, lotions, and solutions. In addition, compositions of the invention may be included/soaked in a facial tissue, towelette, or a skin patch. These compositions may be applied to local areas of skin, such as face, hands, arms, etc., in a manner similar to the application of any other skin care products.

Compositions of the invention have been found to be effective in improving skin hydration or moisture content. Tests on volunteers show that compositions in accordance with embodiments of the present invention can maintain skin moisture with only a few applications. These tests, using Lo-108 as an example, are described in detail below.

### EXPERIMENTAL TESTING

### Test 1

### Efficacy of Lo-108 for Acne Treatment

### Material and Method

This experiment was conducted as an open clinical trial. Between February and August of 2001, 60 test subjects were randomly selected (30 male, 30 female). The average age of the test subjects was 25 years old with a range between 17 and 42 years old. Daily facial treatment of Lo-108 was prescribed for 6 months, with each daily treatment lasting between 3 to 8 hours (overnight). The subjects visited the clinic once every two weeks for evaluation. Details of the treatment procedure are as follows:
1. Facial areas selected for the experiment were first cleaned with soap and then allowed to dry before applying Lo-108.
2. Approximately 2 ml of Lo-108 was applied to the selected facial areas. The selected areas were then covered with swab to prevent oozing.
3. After 3 hours, the swab and Lo-108 were removed without using any lotions or moisturizers.
4. Any pustules were squeezed out before applying the treatment.

If a test subject presented with pustules, oral antibiotics such as tetracycline or vibramycin were prescribed in conjunction with the treatment for one week. Use of other acne treatments such as vitamin A acid (either oral or topical), or hormone therapy, were prohibited during the rest of the trial period. The results were examined by both the physicians and the test subjects themselves. The degree of improvement was assessed by comparing the number of acnes and pustules and the level of inflammation before and after the experiment.

### Results

TABLE 1
Efficacy of Lo-108 in Acne Treatment

| | | After 8-Week |
|---|---|---|
| | Before Treatment | Treatment |
| Mean # | 43.5 | 20.1 |
| Camedones | | (p < 0.01) |
| Mean # Papules | 21.0 | 2.1 |
| | | (p < 0.001) |
| Mean # Pustules | 8.9 | 0 |
| | | (p < 0.0001) |
| Mean # Cysts | 0.8 | 0 |

As shown in Table 11, after 8 weeks of treatment, the number of blackhead and whitehead acnes significantly reduced from an average of 43.5 to 20.1 (p<0.01). Furthermore, the number of red-swelling type acnes due to shrunken pore opening was also significantly reduced from an average of 21.0 to 2.1 (p<0.001). The red-swelling pustules and cysts also reduced from an average of 8.9 and 0.8, respectively, to none, leaving only partial reddish speckles and scars. In fact, the reduction of red-swelling became apparent in the first four weeks of treatment, which encouraged the test subjects to continue to use Lo-108. During the trial, it was observed that in addition to anti-inflammatory, cooling, and swell-reducing properties, Lo-108 also exhibited abilities to reduce excess cutinization and improve smoothness of the skin.

Prior art topical anti-cutinization medication or topical antibiotics often cause side effects such as skin dryness, pain, even swelling. But because Lo-108 also has moisturizing properties, none of these side effects occurred.

Based on the above results, the therapeutic effect of Lo-108 for acne treatment is quite clear. Lo-108 may also have other therapeutic effects of, such as reduction of cutin and sebum secretion, and germicidal and anti-inflammatory functions.

### Experiment 2

### Comparative Study of Excess Sebum Secretion Inhibition

### Material and Method

Twenty healthy test subjects (volunteers) ages between 18 to 55 participated in this experiment. The foreheads of the subjects were first divided into two regions (left and right). Only one region of each subject was selected to received a daily dosage of Lo-108. Each night, about 1 ml-2 ml of Lo-108 were applied to the selected region of the forehead. Lo-108 was left on the forehead for 3 hours before removing. The regions receiving Lo-108 are the testing regions and the regions not receiving Lo-108 are the control regions. This treatment was continued for 4 weeks. Patients were regularly examined once a week for sebum amount on each side of the forehead.

Sebometer 810 PC (Courage and Khazaka Ltd, Germany) was used to measure the amount of sebum. Because the test was designed to measure the percent difference between sebum amount on the left and the right side of the forehead, the effects of interfering factors may be reduced to a minimum.

To measure the amount of sebum, the head of a sebometer, usually a piece of opaque plastic material (0.1 mm×64 mm²), is pressed on the surface of the skin for 30 seconds. During this time, the plastic material absorbs sebum on the skin and becomes less opaque. The change in opaqueness has a linear relationship to the amount of sebum absorbed, so by measuring the optical transmittance of the plastic material, the amount of sebum on the skin (in mg/cm²) can be determined.

### Results

TABLE 2
Time course

| Time | | |
|---|---|---|
| (weeks) | Lo-108 | Control |
| 0 | 88.15 ± 13.02 | 93.85 ± 12.82 |
| 1 | 82.80 ± 12.40 | 94.00 ± 13.52 |
| 2 | 88.00 ± 9.53 | 112.55 ± 12.88 |
| 3 | 81.75 ± 11.24 | 118.8 ± 12.66 |
| 4 | 61.15 ± 8.36 | 99.95 ± 10.38 |

Note:
Twenty subjects with Lo-108 applied to the right cheeks and the vehicle applied to the left cheeks (control).

TABLE 3
Results of Sebum Secretion

| | Degree of | | | | |
|---|---|---|---|---|---|
| Source | Freedom | ANOVA SS | Mean Square | F Value | Pr > F |
| Patients | 19 | 229013.20 | 12053.33 | 6.88 | 0.0001 |
| Treatment | 4 | 11278.55 | 2819.64 | 1.61 | 0.1740 |
| Duration and | | | | | |
| Frequency | | | | | |
| Treatment | 1 | 27518.58 | 27518.58 | 15.71 | 0.0001 |
| Treatment | 4 | 8869.17 | 2217.29 | 1.27 | 0.2854 |
| and Time | | | | | |
| Interaction | | | | | |

Because both the testing region and the control region were located on the same person, this eliminated the effect of variations in temperature, humidity, level of exercise and sweating, and many other extraneous factors. From Table 2, it seems that the weekly average differences appeared to show a trend that is increasingly prominent.

ANOVA analysis of Lo-108 data (as shown in Table 3) demonstrated that sebum amount on the test regions showed a substantial reduction, as compared to the control regions. In fact, reduction of sebum by Lo-108 treatment showed statistical significance within the first week of use (pr>F 0.0001). When the difference in sebum secretion is analyzed as a function of time, it appears that similar results were obtained for longer periods of application time. That is, no significant increase with time was observed (pr>F 0.2854). Thus, based on the above results, Lo-108 is effective in reducing and inhibiting sebum secretion for up to 12 hours, mitigating the accumulation of sebum on the pore openings (the main reason for acne). This effect can be achieved in a short period of time. Although it has not been shown that the more Lo-108 used, the better the inhibitory effect, it has been demonstrated that daily use of Lo-108 may maintain the inhibitory effect for up to four weeks.

### Experiment 3

### Animal Testing of Burn Injury and Wound Healing

### Material and Methods

This experiment used eight-weeks old male Wistar mice. The mice were caged in the Laboratory Animal Center of Nation Cheng-Kung University, the only facility in south Taiwan that meets international SPF (specific pathogen free) standards, and kept in air-conditioned environment at 25±1° C. All mice were allowed to eat and drink freely.

### Burn Injury Experiment

The experimental protocol of this set of experiments followed that of Kistler, and used the animals for self-comparison. The procedure used are as follows:

Each mouse was first sedated with 65 mg/kg of pentobarbital. After ensuring that the animals were sedated, the back of each animal was then divided into four regions, each about 4 cm² in size. The hairs inside each region were shaved off with an electric shaver. A piece of iron heated to 80-85° C. was placed in each of the four regions for approximately 10 seconds to inflict burn injuries. The irons were then removed and the wounds treated with 37% hydrogen peroxide to sterilize the burned areas. After the injuries were made, different treatments were applied to each of the four regions. The four treatments were vitamin E, base solution of the test product, the test product, and no treatment for control. The solutions were applied in sufficient amount to completely cover each of the respective regions. After application of the solutions, the wounds were completely covered to prevent infection. The wound dressing was changed once a day at the same time each day. A photograph was also taken everyday to follow the changes. After seven days, the animals were sacrificed for pathology examination.

### Results

Based on observations of the wounds, the region that was treated with the products according to this invention showed a lesser degree of inflammation.

### Wound Healing Experiment

This experiment also employed eight-weeks old Wistar mice and a similar experimental protocol as the previous experiment. The mice were first sedated. The back of each mouse was then divided into four regions, each about 4 cm² in size. Back hairs were removed with an electric shaver, and an incision of about 1 cm was made in each of the four regions with a surgical knife and sterilized with hydrogen peroxide (37%). The wound was deep enough to reveal the muscle layer. Each of the four regions received a different treatment. The control region received no treatment. The other three regions each received vitamin E, the test product with the base solution, and the base solution, respectively. A sufficient amount of each of the treatments was applied to cover the entire surface of the respective regions. Right after the treatments were applied, the wounds were covered up completely to prevent infection. Wound dressings were changed once a day at regular intervals. A photograph was also taken once a day to record the progress of healing. The healing effect of a treatment was determined by the amount of time required for the treated wound to reach complete healing, i.e., the less time required to reach complete healing, the greater the healing effect of the treatment.

### Results

Based on the rate of healing on the incisions, the regions treated with the test product showed fastest rate of healing, approximately 7.13±1.27 days on average (N=8). In contrast, the control region required 11.00±2.24 days on average (N=8). The rate for the base solution, 10.13±1.62 days (N=8), is nearly the same as that of the control (P>0.05).

### Experiment 4

### Bacteria Inhibition

The microbes used for this experiment includes Staphylococcus aureus, Methicillin Restant (ATCC 33591), Staphylococcus aureus, and Propionibacterium aureus (ATCC 6919). After culturing in suitable culture media, the Lo-110 in concentrations of 0.03 μg/ml, 0.1 μg/ml, 0.3 μg/ml, 1 μg/ml, 3 μg/ml, 10 μg/ml, 30 μg/ml, and 100 μg/ml was applied to the culture. The inhibitory concentration of Lo-110 was determined to be 100 μg/ml.

### Experiment 5

### Skin Moisturization Effect

Twenty-four healthy subjects (12 males, 12 females) between ages 20 and 40 with no history of allergic reactions to skin medications, no dermatological conditions within past 3 months, and no facial scars were selected and randomly separated into two groups. Prior to this experiment, the subjects all agreed to not apply any other substances to the testing areas of their skins before and during the experiment. Sweating and exposure to the sun were also kept to a minimum. The experiment was carried out using the following protocol:

Each of the two groups consists of twelve randomly selected members from the pool of 24 subjects. The first group received Lo-107 while the second group received Lo-108. On each subject, the left side of the face received the designated treatment in amounts of about 1 ml while the right side of the face received no treatment as a control.

The subjects were asked to arrive at the testing location at 4 pm. Upon arrival, the entire facial skin was first cleaned with a neutral cleanser. After waiting for fifteen minutes, the amount of moisture was measured and recorded as the baseline level before applying the test product. Corneometer CM 825 (Courage and Khazaka Ltd, Germany) was used for measuring the moisture level. Then, the test product was evenly applied to the testing side of the face (both cheek and forehead) and left there for about 30 minutes. After the 30 minute waiting period, the test product was wiped off of the face with wet paper towels and cleaned with a neutral cleanser. The moisture level of the skin was measured fifteen minutes after the product was wiped off. Data points were taken from two different locations of each side of the forehead and cheek. The collected data were then analyzed by ANOVA.

### Results

Both Lo-107 and Lo-108 showed weak moisture retaining effects in the forehead regions. However, they both showed significant moisture retaining effects in the cheek regions.

### Experiment 6

### Long Term Skin Hydration

### Material and Method

The test subjects (volunteers) include 12 physically healthy male and female between 20 and 28 years of age. These volunteers had no prior dermal allergy to drugs, no onset of skin illness within three months before the test, and no scar on the forehead and cheek. The volunteers were prohibited from applying any substance to the test areas before and during the tests, and were asked to minimize exposure to sunlight and sweat on forehead and cheek. Each of the test subjects signed a consent before the test.

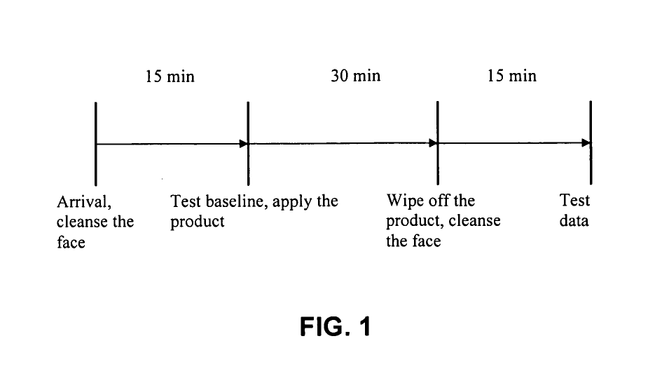

FIG. 1 shows a schematic of a test protocol, which was used to perform the test on a daily basis. The test subjects arrived at the test location at 5 p.m., cleansed their faces with a neutral cleanser. After 15 minutes, a multi-functional skin tester, MPA 5™ (from Courage and Khazaka, Ltd, Germany), was used to record the baseline skin hydration. Then, 1 ml each of Lo-108 lotion (Example 2) was applied to an area of about 15 cm² each on the left forehead and cheek. No lotion was applied on the right side for control. The lotion was allowed to remain on the skin for 30 minutes. After 30 minutes, the residue substance on the skin surface was wiped clean with a wet paper towel. The test subjects again cleansed their faces with a neutral cleanser. After 15 minutes, skin hydration (data) was assessed on the left and right sides of the forehead and cheek of each individual. The above test and data collection were repeated daily for one week.

### Data Analysis

The skin hydration data of the control side and the experiment side for each individual were corrected for the baseline skin hydration that was recorded before the test on day 1. The baseline corrected data, which correspond to changes of skin hydration of the test subjects, were then analyzed for the skin moisturizing effects of the Lo-108 lotion. ANOVA was used to analyze the changes of skin hydration on the control group and the experiment group.

### Results

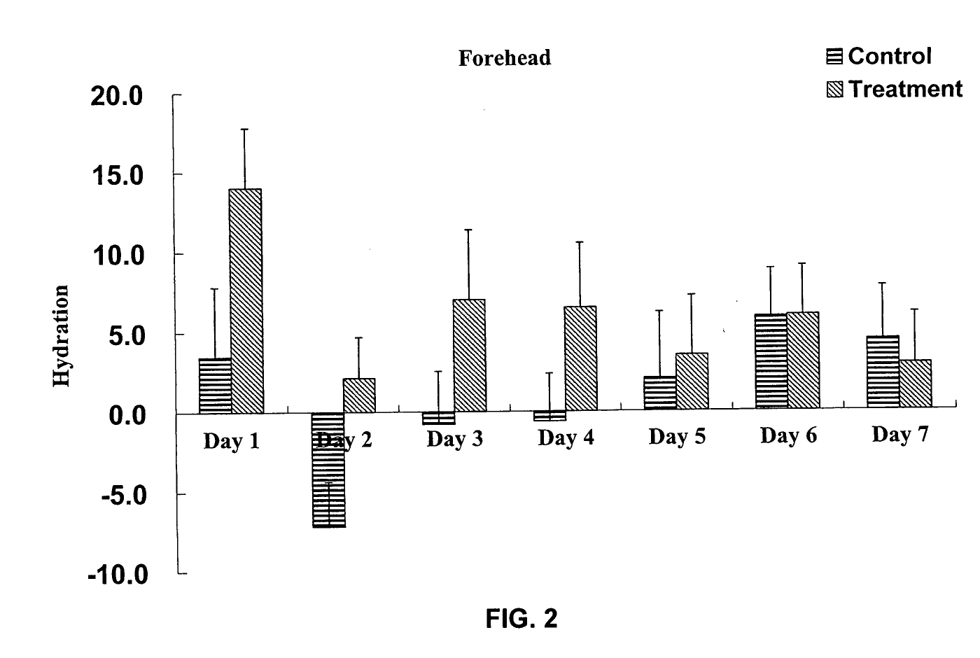

FIG. 2 shows results for tests performed on the forehead. On day 1, the average change in skin hydration of 12 subjects on the right side of the forehead was 3.5 (control group), and 14.0 on the left side of the forehead (experiment group). This result shows that the effect of the skin moisturizing composition is immediate. On the second and third days, further improvement in skin hydration was observed in the experimental group. As the test continued, however, no further improvements in average skin hydration was observed, suggesting that maximal benefits had been achieved and the first few days. The ranges of changes in skin hydration were −7.2˜5.9 for the control group, and 2.1˜14.0 for the experiment group.

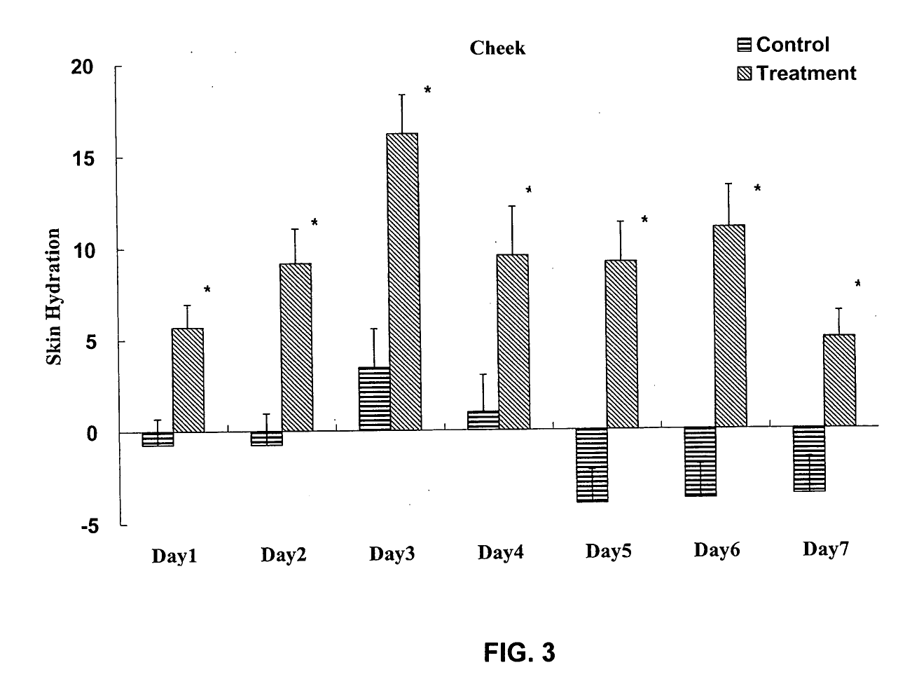

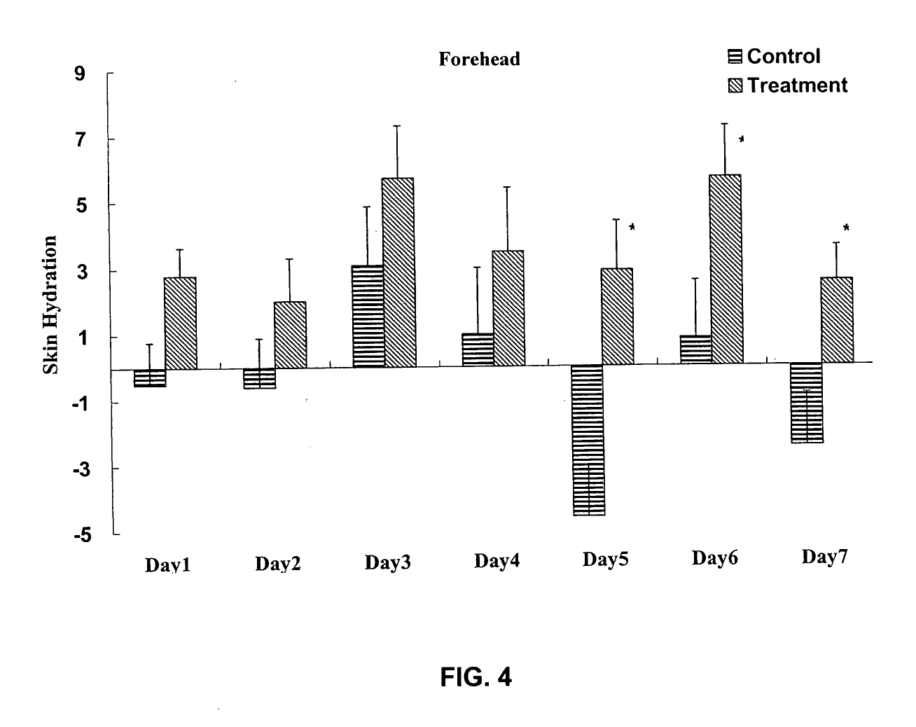

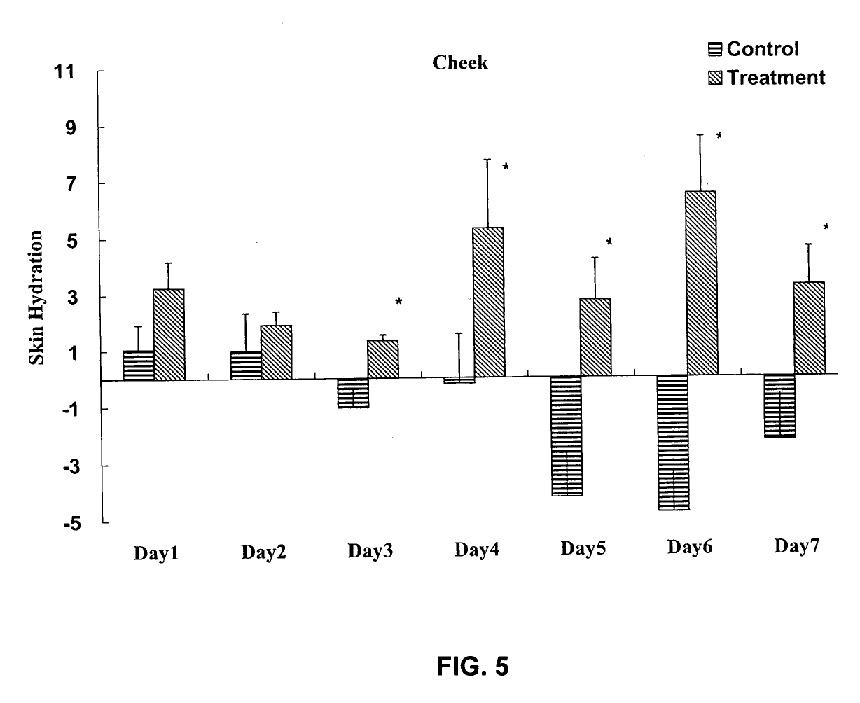

FIG. 3 shows results for tests performed on cheek. On day 1, the average changes in skin hydration of 12 subjects were −0.73 for the control group (right cheek) and 5.68 for the experimental group (left cheek). Again, this result shows that the moisturizing effect was immediate. On days 2 and 3, further improvements in skin hydration were observed in the experimental group. As the test continued, however, no further improvements in average skin hydration were observed, suggesting that maximal benefits of the lotion had been achieved in the first few days. The range of average skin hydration changes for the experiment group was 5.03˜16.21. Similar results are obtained with Lo-107 as shown in FIG. 4 (foreheads) and FIG. 5 (cheeks).

These results show that compositions of the invention are effective in improving skin hydration with only a few applications. Furthermore, the improvement can be maintained by continuing to use the compositions.

Some embodiments of the invention relate to methods for improving skin hydration by using compositions of the invention. In a typical method, a composition of the invention is provided in a suitable form, such as lotions, solutions, creams, etc. Users would apply the composition to areas of skin where improvement in skin hydration is desired. Because the main components of the compositions are vitamins, the compositions shall be safe for long term use. Thus, the users can decide how long to allow the compositions to stay on the skin. In some methods, the compositions may be included in a wet facial tissue or a wet towelette. With these forms, a user can use the tissue or the towelette to wipe the dry areas of the skin. Yet in other embodiments, compositions of the invention may be packed in (or coated on) a patch, which can be placed on the dry skin to improve skin hydration. One of ordinary skill in the art would appreciate that other methods of application may also be used without departing from the scope of the invention.

Advantages of compositions and methods of the invention may include one or more of the following. The skin care compositions of the invention are based on vitamins, which are relatively safe for long term use. The compositions do not include vitamin A acid. Therefore, there is no risk of osteoporosis even after long term use. The skin moisturizing compositions of the invention are effective in treating acnes, zits, comedo, or skin dryness.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

What is claimed is:

1. A composition for local application, comprising:
vitamin C in an amount from about 1% to 45% w/w;
vitamin B complex in an amount from about 1% to 5% w/w;
carotene in an amount from about 0.1% to 3% w/w; and
vitamin E in an amount from about 2% to 90% w/w.

2. The composition of claim 1, wherein the composition further comprises a thickening agent in an amount from about 1% to 5% w/w.

3. The composition of claim 1, wherein the composition further comprises a surfactant in an amount from about 1% to 8% w/w.

4. The composition of claim 1, wherein the composition further comprises a fragrance in an amount from about 0.1% to 2% w/w.

5. The composition of claim 1, wherein the vitamin B complex comprises at least two selected from the group consisting of vitamin B1 (thiamin), vitamin B2 (riboflavin), niacin, vitamin B5 (pantothenic acid), vitamin B6 (pyridoxal), folic acid, vitamin B12 (cobalamin), and biotin.

6. The composition of claim 1, wherein the vitamin C is in an amount from about 4% to 15% w/w.

7. The composition of claim 1, wherein the vitamin B complex is in an amount from about 1% to 3% w/w.

8. The composition of claim 1, wherein the carotene is in an amount from about 0.1% to 2% w/w.

9. The composition of claim 1, wherein the vitamin E is in an amount from about 15% to 65% w/w.

10. The composition of claim 1, wherein the vitamin C is in an amount from about 4% to 15% w/w, the vitamin B complex is in an amount from about 1% to 3% w/w, the carotene is in an amount from about 0.1% to 2% w/w, and the vitamin E is in an amount from about 15% to 65% w/w.

11. The composition of claim 1, wherein the skin care product is in a form selected from a lotion, an ointment, a cream, a solution, and a paste.

12. The composition of claim 1, wherein the skin care product is in a form selected from a tissue, a towelette, and a patch.

13. The composition of claim 1, wherein the vitamin C is in an amount about 4% w/w, the vitamin B complex is in an amount about 1% w/w, the carotene is in an amount about 1% w/w, and the vitamin E is in an amount about 65% w/w.

14. The composition of claim 13, wherein the composition further comprises a fragrance in an amount about 1% w/w, a surfactant in an amount about 6.5%, and a thickening agent in an amount about 4%.

15. Use of the composition of claim 1 in the manufacturing of a skin care product for treating acnes, zits, comedo, or skin dryness.

16. A method of treating a skin condition, comprising applying a composition of claim 1 to an area of skin.

17. The method of claim 16, wherein the skin condition is at least one selected from acnes, zits, comedo, and skin dryness.
